(19) United States
(12) Patent Application Publication
(10) Pub. No.: US 2017/0042254 A1
(43) Pub. Date: Feb. 16, 2017

(54) DISPOSABLE UNDERGARMENT AND RELATED DISPOSAL METHODS

(71) Applicants: Fredrica V. Coates, Winston-Salem, NC (US); Rachel H. Star, Chicago, IL (US)

(72) Inventors: Fredrica V. Coates, Winston-Salem, NC (US); Rachel H. Star, Chicago, IL (US)

(21) Appl. No.: 14/825,945

(22) Filed: Aug. 13, 2015

(51) Int. Cl. A41B 9/00; A61F 13/62; A61F 13/505

(52) U.S. Cl. CPC A41B 9/008; A61F 13/505; A61F 13/627; A41B 2500/10; A41B 2400/52

(57) ABSTRACT

An undergarment includes at least one panel of stretchable loop material. The panel of stretchable loop material is connected to a main body of the undergarment such that it can stretch around one side of wearer's waist or hip. The stretchable loop material is configured such that it can releasably connect to a plurality of engaging elements. The engaging elements can be hooks of a hook-and-loop fastening system. The undergarment is configured such that it can be easily concealed beneath clothing and can be easily positioned and removed by a wearer. A method of disposing an undergarment includes rolling or folding the main body of the undergarment and tying panels of stretchable loop material around the rolled or folded portion to encase the rolled or folded portion between the panels of stretchable loop material.

### BACKGROUND

Disposable undergarments are often used by individuals in order to capture urinary leakage attributable to incontinence. Incontinence can be attributed to a variety of medical conditions. Individuals with such conditions may use disposable undergarments to capture involuntary urinary leakage. Such individuals require disposable undergarments that can be worn under clothing.

Known disposable undergarments suffer from many disadvantages. Known disposable undergarments are often bulky, poorly fitting, inconvenient for use in an active lifestyle and create problems during disposal. Many known disposable undergarments are of the pull-on type. As such, the disposable undergarment may require a wearer to remove his or her pants in order to remove and replace the disposable undergarment. This can be a time-consuming and cumbersome activity that does not work well for individuals with active or busy lifestyles. In addition, known disposable undergarments are often cumbersome due to the fact that they are designed to retain both solid and liquid wastes and are also designed to be one-size-fits-all products. Known undergarments do not conform to a wearer's body and can be seen through a wearer's clothing due to the attachment schemes, bulky absorbent cores and general over-engineering of the products. There exists a need, therefore, for a disposable undergarment that can be easily concealed, easily removed and replaced during normal daily life and that can accommodate a variety of body types and sizes.

Known disposable undergarments also can be difficult to dispose of easily and sanitarily after they have become soiled. It can be difficult to retain the captured wastes in the soiled undergarment once it has been removed from a wearer. There exists a need for a disposable undergarment that can retain bodily wastes and can be packaged or configured to retain such wastes from a point of removal to a point of disposal.

### BRIEF SUMMARY

The instant disclosure describes various embodiments of an undergarment. In a first embodiment, an undergarment includes at least one panel of stretchable loop material. The panel of stretchable loop material is connected to a main body of the undergarment such that it can stretch around one side of a wearer's waist or hip. The stretchable loop material is configured such that it can releasably connect to a plurality of engaging elements. The engaging elements can be hooks of a hook-and-loop fastening system. The stretchable loop material can be a knit fabric that includes spandex yarn.

In a second embodiment, the undergarment includes a first panel and a second panel of stretchable loop material connected to opposite sides of the main body of the undergarment.

In a third embodiment, the height of the first flap is at least twice as long as its width.

In another embodiment, the undergarment includes a main body with an absorbent core with a first and second longitudinal side. A first tab is connected to the main body on the first longitudinal side and includes a first attachment area with a plurality of engaging elements. The undergarment also includes a first flap extending from the main body on the first longitudinal side. The first flap is made of stretchable loop material that is configured to releasably and directly connect to the first tab by engaging with the plurality of engaging elements.

In another embodiment, any location on the first flap's body-facing surface can engage with the plurality of engaging elements.

In another embodiment, the first tab and the first flap are configured to permit the undergarment to be worn by wearers with variations in weight of over 20 pounds.

In another embodiment, the undergarment further includes a second tab and a second flap both connected to the main body on the second longitudinal side. The second flap is made of stretchable loop material configured to releasably and directly connect to the second tab by engaging with a plurality of engaging elements on the second tab.

### BRIEF DESCRIPTION OF THE SEVERAL VIEWS OF THE DRAWINGS

Certain embodiments are shown in the drawings. However, it is understood that the present disclosure is not limited to the arrangements and instrumentality shown in the attached drawings, wherein:

FIG. 1 is an illustration of one embodiment of a disposable undergarment of the present disclosure.

FIG. 2 is an illustration of the garment-facing side of the embodiment of FIG. 1.

FIG. 3 is a side view of one embodiment of a disposable undergarment of the present disclosure on a wearer.

FIG. 4 is a front view of one embodiment of a disposable undergarment of the present disclosure on a wearer.

FIG. 5 is an illustration of another embodiment of a disposable undergarment of the present disclosure.

FIG. 6 is an illustration of the garment-facing side of the embodiment of FIG. 5.

FIG. 7 is an illustration of one embodiment of the undergarment in the process of being prepared for disposal.

FIG. 8 is an illustration of one embodiment of the undergarment in a tied position ready for disposal.

### DETAILED DESCRIPTION

For the purposes of promoting and understanding the principles disclosed herein, references are now made to the preferred embodiments illustrated in the drawings and specific language is used to describe the same. It is nevertheless understood that no limitation of the scope of the invention is thereby intended. Such alterations and further modifications in the illustrated device and such further applications of the principles disclosed as illustrated herein are contemplated as would normally occur to one skilled in the art to which this disclosure relates.

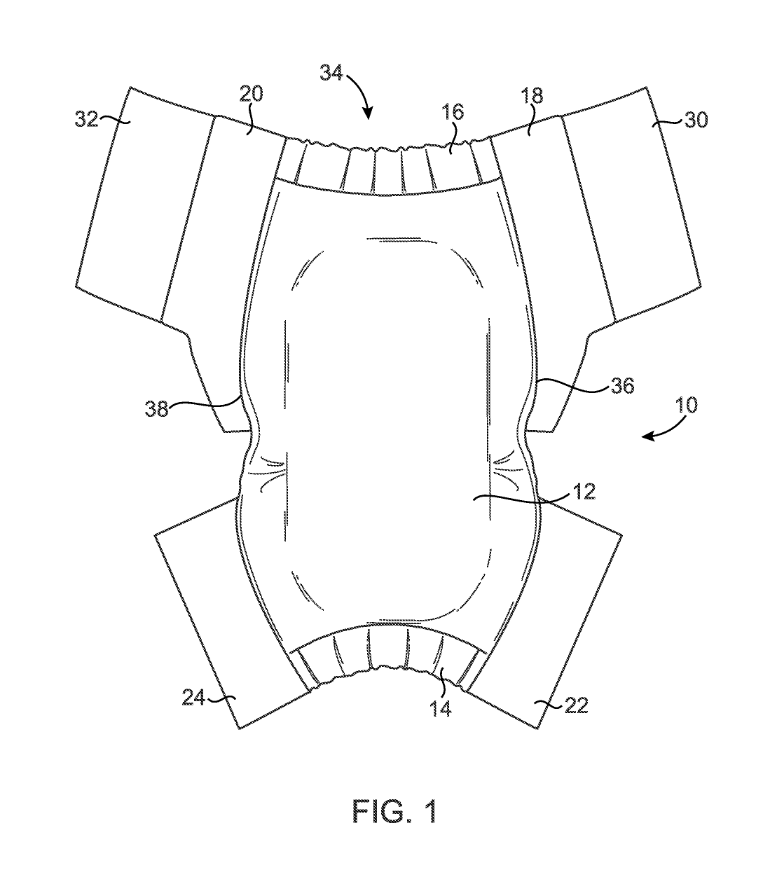

FIGS. 1-4 show one embodiment of disposable undergarment 10. Disposable undergarment 10 may include a main body 34, first and second extensions 18, 20, first and second tabs 22, 24 and first and second flaps 30, 32. Disposable undergarment is generally shaped and contoured to be worn close to a wearer's body such that it can be concealed while still capturing leaks, bodily fluids and waste. Main body 34 is the central portion of disposable undergarment 10 and includes absorbent core 12 that serves the purpose of capturing fluid and preventing its escape from disposable undergarment 10. Various structures and layers of materials can be used to construct main body 34 and absorbent core 12.

The main body may include a top sheet and one or more backsheets. The layers of material can be paper, plastic or other suitable natural or synthetic fabrics. Such materials can be suitable nonwoven materials. The materials closest to a wearer's body are often made of breathable or permeable materials such that wastes are permitted to pass through such materials and are kept away from a wearer's skin. Other outer layers and materials can be made from impermeable materials such that bodily wastes are not permitted to escape or otherwise leak from the undergarment. Elastic bands or other materials can be fixed between the layers of material or to the layers of material to provide elasticity, gathers or other features to conform the disposable garment 10 to the body of a wearer. The absorbent core 12 may include one or more acquisition and liquid absorption layers that are intended to capture and hold bodily fluids. Known methods of connecting the various layers of main body 34 may be used to construct disposable undergarment 10 such as thermal bonding, adhesive, ultrasonic sealing and various stitching methods.

Main body 34 may also include front waist portion 14 and rear waist portion 16. As can be appreciated, in use, front waist portion 14 is configured to fit against the front of a wearer's waist area. Rear waist portion 16 is configured to fit against the rear waist of a wearer under the wearer's lower back. As stated earlier, disposable undergarment 10 is low-rise such that it can be concealed under a wearer's clothing. As such front waist portion 14 and rear waist portion 16 are configured to be positioned on a wearer's body under and away from the waist region of wearer's clothing. Front waist portion 14 and rear waist portion 16 may include elastic bands, elastic strips or other features to contour disposable undergarment 10 against the body of a wearer. Furthermore, the elasticity provided at front waist portion 14 and rear waist portion 16 can provide the benefit of permitting disposable undergarment 10 to be worn by wearers of various shapes and sizes while still reliably capturing and retaining bodily fluids. Elastic bands, strips or other extensible members can be embedded between layers of main body 34 at front waist portion 14 and rear waist portion 16 or can be connected at these locations via various methods of attachment, as discussed above, such as thermal bonding, adhesive, ultrasonic sealing or stitching.

Along the longitudinal sides of main body 34 (i.e., in a direction extending between front waist portion 14 and rear waist portion 16) various members or panels may be attached to assist with the attachment of disposable undergarment 10 to the body of a wearer. In the example shown in FIGS. 1-4, disposable undergarment 10 includes first and second extensions 18, 20, first and second flaps, 30, 32 and first and second tabs 22, 24. First and second extensions 18, 20 are connected to main body 34 near rear waist portion 16 and are further connected to main body 34 along the longitudinal sides of main body 34 from waist portion 16 toward the center of main body 34. First and second extensions 18, 20 can be separate pieces attached to main body 34 or can be extensions of one or more layers of the material that is used to construct the main body of disposable undergarment 10. First and second extensions 18, 20 are preferably made of an elastic material such as an extensible laminate or a breathable partial laminated nonwoven material. Other suitable materials may also be used.

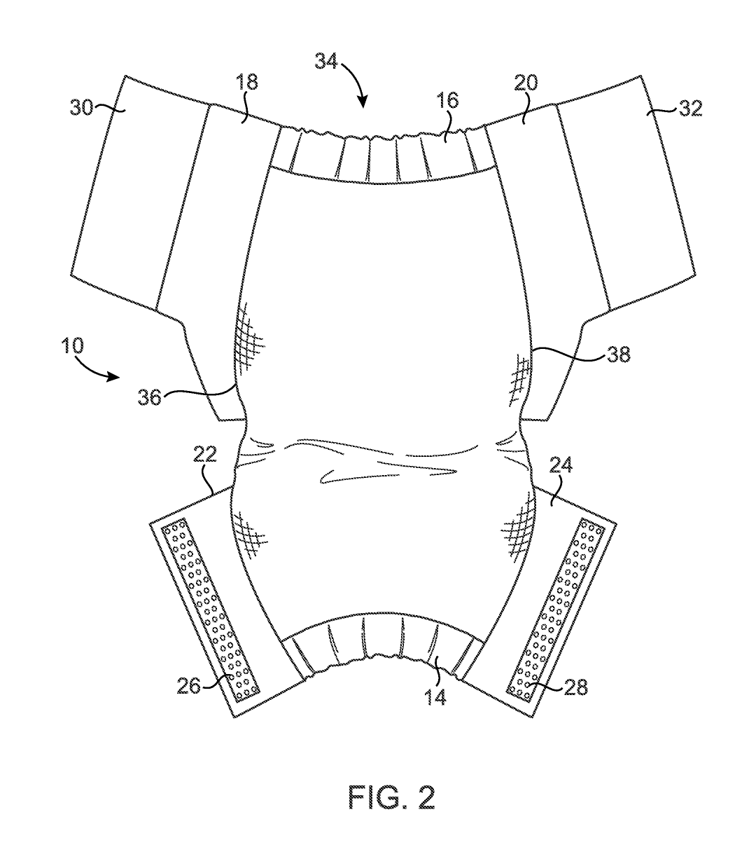

As can be seen in FIGS. 1 and 2, in this example, first and second extensions 18, 20 are connected to main body 34 at a proximal end of first and second extensions 18, 20. At distal ends of first and second extensions 18, 20, first and second flaps 30, 32 are connected. First and second flaps 30, 32 are second pieces of material connected to and extending in a transverse direction away from main body 34. First and second flaps 30, 32 have a width that is measured in a direction that is generally parallel to a top edge of rear waist portion 16 and a height that is measured in a direction that is generally perpendicular to the width. In this example, the height of first flap 30 is at least approximately twice as long as the width. While various sizes of disposable undergarment 10 can be constructed, in one example configuration, first and second flaps 30, 32 are approximately 55-65 mm wide and 100-130 mm long. Other sizes and shapes of first and second flaps 30, 32 can also be used.

First and second flaps 30, 32 can be connected to first and second extensions 18, 20 via any suitable means. Suitable methods of attachment may include, but are not limited to, adhesive, stitching, thermal bonding, ultrasonic sealing and the like. In one preferred embodiment, first and second flaps 30, 32 are secured via an overlock stitch to first and second extensions 18, 20. First and second flaps 30, 32 are preferably made of a stretchable loop material. Stretchable loop material is elastic and provides the advantage of contouring the flaps to a wearer's body and accommodating a wide variety of wearer body types and shapes. In one example, first and second flaps 30, 32 are made of a stretchable loop material that is a woven or knit material that is capable of being extended to at least twice its initial length. In other examples, the stretchable loop material can be extended to 220% of its original, non-extended length. Stretchable loop material additionally has the property that it can securely engage to the hook portion of a typical hook-and-loop fastening system. In this manner, and as will be described, first and second flaps 30, 32 can directly engage to an engaging element of a hook-and-loop fastening system. This property allows first and second flaps 30, 32 to be made of a single material without the need for manufacturing the flaps from multi-layers of material or to connect a separate fastening mechanism or a separate piece of loop material to the flap. This property of the stretchable loop material of first and second flaps 30, 32 also permits the engaging of the flaps at any position along its entire surface. The stretchable loop material permits such engagement, disengagement and re-engagement a significant number of times. These properties provide a significant benefit in providing flexibility, adjustment and comfort for a wearer.

Stretchable loop material with the properties described above can be constructed of different materials using different types of fabrics and threads. In one example stretchable loop material, the material is formed from a stitchbonded fabric that includes a nonbonded fibrous layer in which elastic yarns, such as spandex elastomeric yarns, are stitched to create rows in the fabric with differing abilities to stretch. In this example, the stretchable loop material provides for stretch of 190% in one row and for stretch of 60% in a second row. Substantially perpendicularly from the direction of stretch described above, this example stretchable loop material also provides for 80% to 90% stretch in a second direction as well. This example stretchable loop material has a significant overall stretch in the first direction as well as no more than 90% stretch in a substantially perpendicular direction.

In a preferred embodiment of the above example stretchable loop material, two types of threads are knitted into the substrate fabric. A first spandex thread that is covered with a texturized nylon can be used in addition to a second polyester thread. The first spandex thread with a texturized nylon cover is knitted into the substrate fabric at predetermined intervals in one direction using a simple chain stitch. The second polyester thread can then be knitted into the substrate fabric in one direction using a tricot stitch. These two types of threads can give the stretchable loop fabric a striped or banded appearance.

As discussed above, one of the threads or yarns used in the stretchable loop material can be textured. Textured yarn or thread is subjected to a procedure to increase the volume and the elasticity of the thread or yarn. Textured yarn exhibits twists, crimps, or other distortions along its filaments. In addition to increased elasticity, textured yarns and threads can impart other advantageous properties to fabrics in which it is woven, stitched or otherwise used. One such advantage can be the ability to engage with the hook portion of a hook-and-loop fastening system. The twists, crimps or other distortions give the fabric into which the textured yarn or thread is incorporated a “fuzziness” that can increases its ability to engage and re-engage the hook of a hook-and-loop fastening system.

In one example of disposable undergarment 10, first and second flaps 30, 32 are made of a stretchable loop material such as a knitted fabric of the same construction as a bedskirt knitted fabric available from Xymid, LLC of Midlothian, Va. Another example of a stretchable loop material is a polyester knitted fabric sold as a cover for a mattress by Sinomax USA, Inc. of Houston, Tex. These materials are examples of materials that provide flexibility and extendibility and are able to securely engage to the hook portion of a hook-and-loop fastening system.

In another preferred example of stretchable loop material, the fabric is a knitted fabric using spandex elastic yarn combined with polypropylene. The example stretchable loop fabric may also have one or more layers that can be knitted from the same type of yarn or from different types of yarns, such as from yarns of polyester. Another example knitted fabric stretchable loop material is made of polyester, nylon and spandex yarns. Other suitable woven and knit materials can also be used. Such suitable stretchable loop material's capability to engage the hook portion of a hook-and-loop fastening system can be further improved by subjecting the material to heat treatment prior to manufacturing of the disposable undergarment. Such heat treatment can include repeated washing and drying of the material on high heat prior to manufacturing of the disposable undergarment. Heat treatment of the material can increase the force required to disengage the material from a hook portion of a hook-and-loop fastening system.

As further shown in FIGS. 1-4, disposable undergarment 10 can also include first and second tabs 22, 24. First and second tabs 22, 24 are connected to main body 34 at a location at or near front waist portion 14. First and second tabs 22, 24 may be elongated pieces of material as shown and may extend longitudinally along the longitudinal sides of main body 34 from front waist portion 14 toward the center of main body 34. In one example, the length of first tab 22 can be generally the same size as the height of first flap 30. First and second tabs 22, 24 can be separate pieces of material joined to main body 34 or can be extended portions of the one or more layers of material that are used to construct main body 34. In one example, first and second tabs 22, 24 are pieces of flexible material such as an extensible laminate. Other suitable materials may also be used.

As further shown in FIGS. 1 and 2, first and second tabs 22, 24 may include first and second attachment areas. First and second attachment areas are portions of first and second tabs 22, 24 to which first and second flaps 30, 32 can be connected. In one example, first and second attachment areas 26, 28 can be hook portions of a typical hook-and-loop fastening system. A piece of a hook portion of a hook-and-loop fastening system can include a base with a plurality of engaging elements extending therefrom. This piece of a hook portion can be connected to first and second tabs 26, 28 via any suitable attachment method such as, but not limited to, adhesive, thermal bonding, ultrasonic sealing or stitching. In one example, first and second attachment areas are connected to and extend along a distal edge of first and second tabs 22, 24. As shown in FIG. 2, the engaging elements of first and second attachment areas 26, 28 are located on the garment-facing surface of first and second tabs 22, 24 that is on the surface that faces the clothes of a wearer and away from the body of a wearer. In this manner, first and second attachment areas 26, 28 are exposed when disposable undergarment 10 is positioned on a wearer and are readily available for a wearer to secure first and second flaps 30, 32.

In one example, first and second attachment areas are pieces of hook material from a hook-and-loop fastening system that are approximately 10-15 mm wide by 90-100 mm long. In other examples of disposable undergarment 10, first and second tabs 22, 24 may include one or more first and second attachment areas. Instead of or in addition to first and second attachment areas 26, 28, as shown on FIG. 2, first and second tabs 22, 24 may include a plurality of attachment areas. Several individual pieces of the hook portion of a hook-and-loop fastening system can be connected to first and second tabs 22, 24. The addition of more than one attachment area can provide the advantage of increased versatility and permit disposable undergarment 10 to be fitted to a variety of body shapes and sizes. In still another example, additional attachment areas can be placed on main body 34 at the garment-facing side of main body 34 to provide additional areas where first and second flaps 30, 32 can directly attach. The attachment areas can be in a variety of shapes and patterns so as to provide further variations on the fit and positioning of disposable undergarment 10.

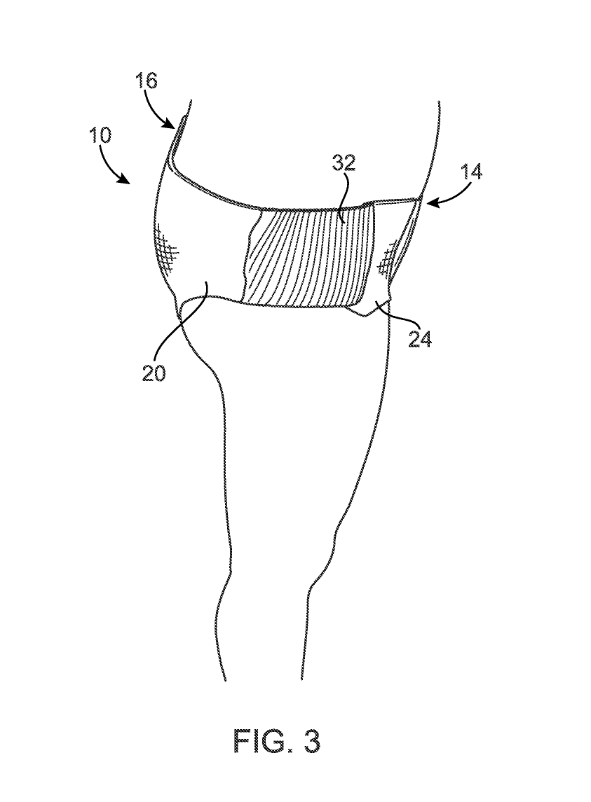

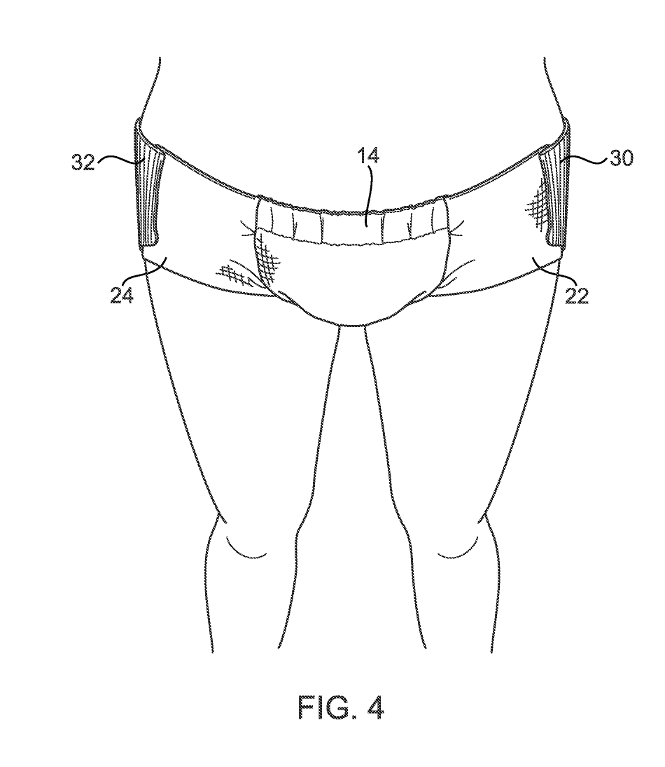

FIGS. 3 and 4 show one embodiment of disposable undergarment 10 secured to a wearer. As can be seen, disposable undergarment 10 is a low-profile or low-rise product, such that it can easily be concealed beneath clothing. Additionally, it is removable, disposable and capable of fitting a wide variety of wearers including wearers, whose respective weights may differ by 20 pounds or more. When secured on a wearer, disposable undergarment 10 wraps around the waist and hip area of a wearer. In doing so, the upper edges of first and second extensions 18, 20, first and second flaps 30, 32 and first and second tabs 22, 24 coupled with the upper edges of front waist portion 14 and rear waist portion 16 circumferentially encompass the waist area of the wearer. The main body portion 34, the upper edges of front waist portion 14 and rear waist portion 16 occupy a relatively small portion of the circumferential waist of disposable undergarment 10. The majority (or more than half) of a wearer's waist is not touched by the main body 34. In other embodiments and sizes of undergarment 10, the main body portion 34 can occupy even less of the circumferential waist of disposable undergarment 10. In one such example, the main body occupies only one-third of a wearer's waist. The majority of a wearer's waist is wrapped by the first and second extensions 18, 20, first and second flaps 30, 32 and first and second tabs 22, 24. The extensible nature of these elements provides a form-fitting and unobtrusive undergarment. This structure of disposable undergarment 10 also provides the flexibility to secure to wearers of a variety of sizes and shapes.

As previously described, the material of first and second flaps 30, 32 is configured of stretchable loop material such that it can directly secure to first and second attachment areas 26, 28. No separate piece of loop material of a hook-and-loop fastening system is required. The material of first and second flaps 30, 32 can directly secure to first and second attachment areas 26, 28 at any location along their surfaces. This also adds to the flexibility, fit and reliability of disposable undergarment 10. A wearer need only hold main body 34 and/or first tab 22 in position and pull or stretch first flap 30 into position and apply pressure at first attachment area 26 to secure one side of disposable undergarment 10 in position. This can be repeated on the second side of disposable undergarment 10 to fix the garment in position. For smaller or thinner wearers, first or second flap 30, 32 can be pulled into an overlapping position for a tighter fit. In such a situation, the distal end of first or second flap 30, 32 would extend over or past first and second attachment area 26, 28 such that first or second flap 30, 32 is engaged at a location on first or second flap 30, 32 nearer to its connection to first or second extension 18, 20. The fact that the stretchable loop material can engage the engaging elements at any location on its surface enables the undergarment to be worn by a wider variety of wearers including wearers with variations in weight of more than twenty pounds.

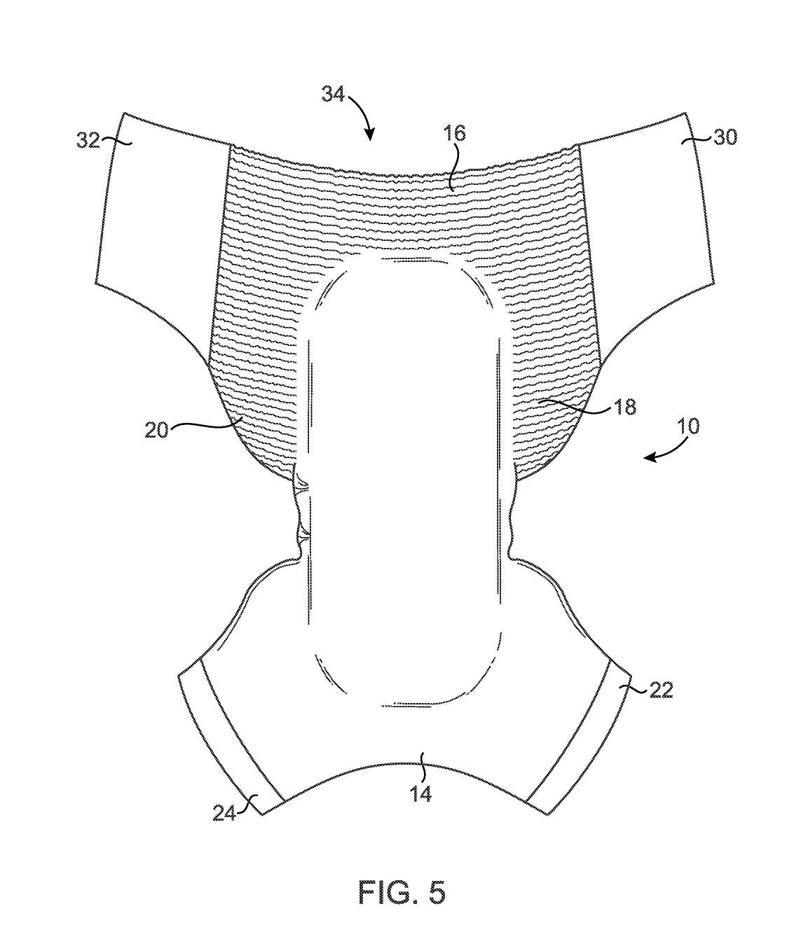

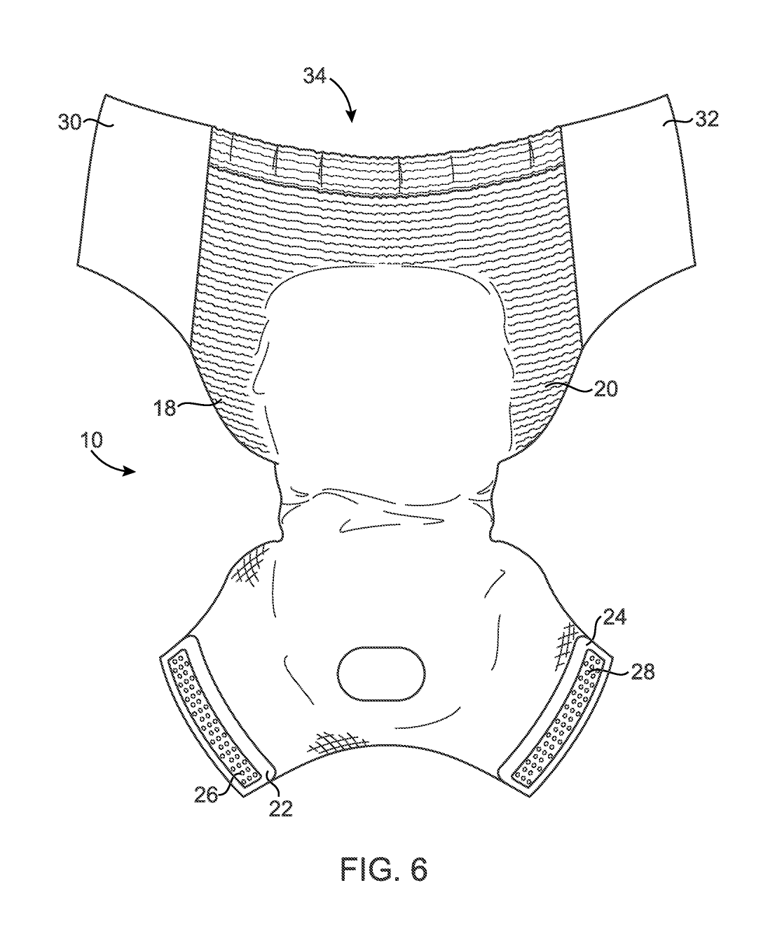

FIGS. 5 and 6 show a second embodiment of disposable undergarment 10. As can be appreciated, many of the same elements are included in this embodiment such as first and second extensions 18, 20, first and second flaps 30, 32 and first and second tabs 22, 24. Front and rear waist portions 14, 16 are also shown as well as main body 34 and absorbent core 12. However, as can be seen, in this embodiment, front waist portion 14 is more curved in shape as are some of the edges of first and second extensions 18, 20, first and second flaps 30, 32 and first and second tabs 22, 24. Various modifications can be made to the relative sizes and shapes of the various elements described herein in an effort to conform disposable undergarment 10 to various shapes and sizes of wearers.

In still other embodiments of disposable undergarment 10, the various layers, flaps and attachment components may include other variations of the elements described above and can include variations in which the panels and attachments may be reversed from front to back or back to front. In another example, first and second extension 18, 20 and first and second flap 30, 32 may all be made of stretchable loop material. The stretchable loop material can also be a continuous piece of material that extends from the main body 34 across the buttocks of a wearer and extend beyond the first and second sides 36, 38 of main body 34 to create wrap portions that can be releasably attached to first and second tabs 22, 24. In this example, since the wrap portions are made of stretchable loop material, they can be directly connected to a hook portion of a hook-and-loop fastening system. In other examples, first and second tabs 22, 24 can also be made of a stretchable loop material. Embodiments of disposable undergarment 10 wherein first and second tabs 22, 24 are made of stretchable loop material can enable the undergarment to fit a wider range of body sizes such as wearers with larger waistlines. As explained above, the stretchable loop material may also extend into or over portions of main body 34 at the front waist region. Such configurations that use more of the stretchable loop material for portions of main body 34 as well as for fastening the disposable undergarment around the waist and hips of wearer can enable a tighter and more body-conforming undergarment.

Disposable undergarment 10 also has an improved design for easier and more sanitary disposal. Once a disposable undergarment captures bodily fluids or other wastes, it must be disposed. Some existing designs of disposable undergarments permit leakage of bodily fluids or other wastes, either during use or upon removal and disposal. Disposable undergarment 10 or the present disclosure permits a wearer to remove the used undergarment and secure the bodily fluids in the absorbent core for easy and sanitary disposal.

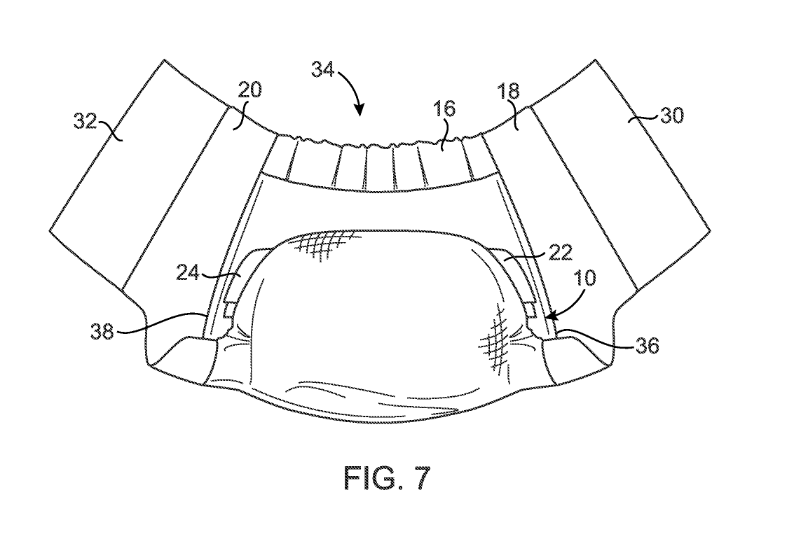

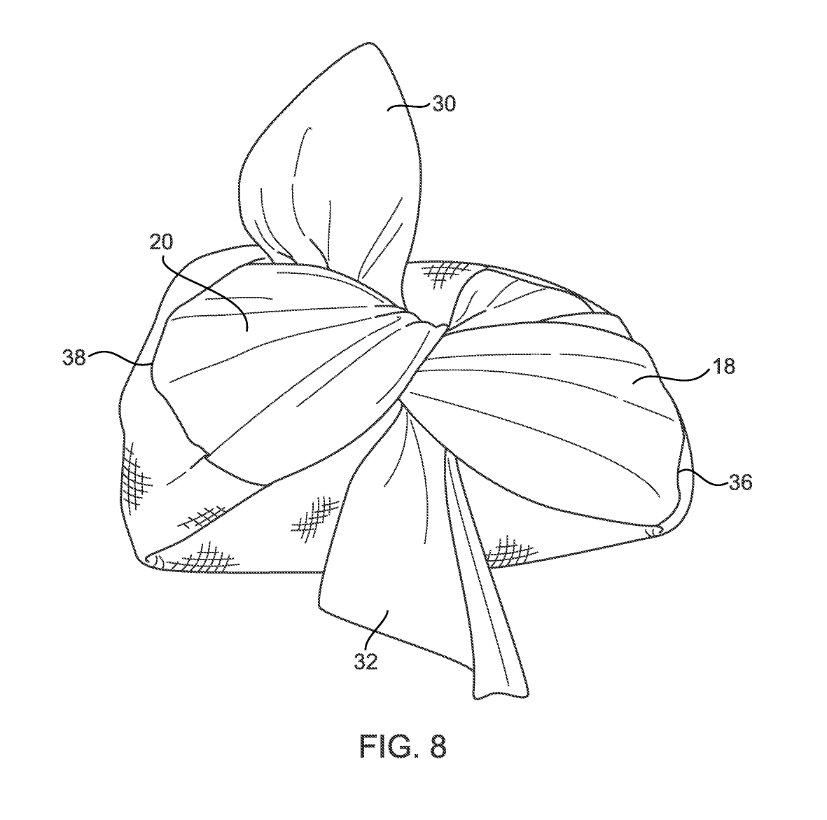

As shown in FIGS. 7 and 8, in order to dispose disposable undergarment 10, the front waist portion 14 can be rolled, folded or otherwise compressed toward the center of disposable undergarment 10 thus capturing the bodily fluid or waste in absorbent core 12 As shown in FIG. 7, front waist portion 14 is compressed, rolled or folded into a position substantially between first and second flaps 30, 32. First and second flap 30, 32 can then be stretched and tied with an overhand or other suitable knot toward each other. In this manner, rear waist portion 16 and first and second flaps 30, 32 wrap around and encase front waist portion and the compressed absorbent core 12 to secure the undergarment in this position for easy and sanitary disposal. The stretchable loop material of first and second flap 30, 32 has the elasticity to encompass the compressed absorbent core and to remain in a tied position as shown in FIG. 8. As can be appreciated, in other alternate embodiments of disposable undergarment 10 wherein more portions of main body, first and second extension or first and second tab are made of stretchable loop material, additional methods of disposal can also be used that wrap and tie the stretchable loop material around a compressed portion of disposable undergarment 10.

In other embodiments of the present disclosure, the undergarment is reusable and not disposable. One or more of the materials can be constructed of natural or synthetic fabrics. The reusable undergarment makes use of the stretchable loop material on first and second flaps 30, 32 that are attached to the other reusable fabrics that are used to construct the various other portions of the reusable undergarment. In this manner, the low-rise, non-bulky aspects of the undergarment as previously described can be maintained in a reusable form. The stretchable loop material on first and second flaps 30, 32 is able to directly engage the hook portion of a hook-and-loop fastening system on or near a front waist portion of the reusable undergarment.

While the particular preferred embodiments have been shown and described, it is obvious to those skilled in the art that changes and modifications may be made without departing from the teaching of the disclosure. The matter set forth in the foregoing description and accompanying drawings is offered by way of illustration only and not as limitation. The actual scope of the disclosure is intended to be defined in the following claims when viewed in their proper perspective based on the related art.

1. An undergarment comprising:
at least one panel of stretchable loop material, wherein the stretchable loop material is operable to releasably connect to a plurality of engaging elements.

2. The undergarment of claim 1 wherein the plurality of engaging elements are hooks of a hook-and-loop fastening system.

3. The undergarment of claim 1 further comprising a main body, wherein the at least one panel of stretchable loop material is operably connected to the main body.

4. The undergarment of claim 3 wherein the at least one panel of stretchable loop material comprises a first panel and a second panel of stretchable loop material operably connected to opposite sides of the main body.

5. The undergarment of claim 4 further comprising at least one attachment area operably connected to the undergarment on an end of the main body opposite the first and second panels of stretchable loop material, wherein the at least one attachment area includes the plurality of engaging elements.

6. The undergarment of claim 5 wherein the at least one attachment area comprises a first and second attachment area operably connected to opposite sides of the main body.

7. The undergarment of claim 1 wherein the stretchable loop material is configured to directly and releasably connect to the plurality of engaging elements at any location along its surface.

8. The undergarment of claim 1 wherein the stretchable loop material is a knitted fabric including spandex yam.

9. The undergarment of claim 1 wherein the stretchable loop material is a stitchbonded fabric including at least one textured thread.

10. A disposable undergarment comprising:
a) a main body having an absorbent core, a first longitudinal side, and a second longitudinal side, each of said first and second longitudinal sides being located laterally outboard of said absorbent core, said main body having a liquid permeable top sheet which contacts a wearer's body and a liquid impermeable backsheet spaced away from said wearer's body, a front waist portion and a rear waist portion, said front and rear waist portions configured to be positioned on a wearer's body under and away from a waist region of said wearer's clothing;
b) first and second elastic extensions each having a proximal end and a distal end, said first elastic extension connected at said proximal end to said first longitudinal side of said main body and said second elastic extension connected at said proximal end to said second longitudinal side of said main body, each of said first and second elastic extensions positioned from a top edge of said rear waist portion to a center of said main body;
c) a first flap and a second flap, said first flap connected to said distal end of said first elastic extension and said second flap connected to said distal end of said second elastic extension, said first and second flaps extending in a transverse direction away from said main body, each of said first and second flaps having a body facing surface which faces said wearer's body, and each of said first and second flaps are constructed of a stretchable loop material that can stretch in two directions and to 220% in a single direction;
a first tab and a second tab, said first tab connected to said first longitudinal side of said main body and said second tab connected to said second longitudinal side of said main body, said first tab having a first attachment area with a plurality of engaging elements and said second tab having a second attachment area with a plurality of engaging elements, each of said first and second tabs longitudinally extending from an edge of said front waist portion toward said center of said main body, and said plurality of engaging elements on said first arid second tabs are engagable with said first and second flaps to secure said disposable undergarment around a wearer's waist and under and away from a waist region of said wearer's clothing.

11. The disposable undergarment of claim 10 wherein each of said front and rear waist portions contain elastic bands, each of said first arid second tabs having a garment facing surface, each of said first and second attachment areas are secured to one of said garment facing surfaces, and each of said body-facing surfaces of said first and second flaps directly engages with said plurality of engaging elements formed on said first and second tabs.

12. The disposable undergarment of claim 10 wherein each of said first and second flaps has a height measured along a length of said main body and a width measured perpendicular to said height, and said length is at least twice said width, said first and second flaps are constructed from a stretchable loop material, said stretchable loop material is formed from a stitch bonded fabric that includes a non-bonded fibrous layer in which elastic yarns are stitched to create rows in the fabric with differing abilities to stretch, and any location on said body-facing side of said first and said second flaps can engage said plurality of engaging elements formed on said first and second tabs.

13. The disposable undergarment of claim 10 wherein said first and second flaps are constructed from a woven, elastic stretchable loop material that is capable of being extended to at least twice its non-extended length, said plurality of engaging elements formed on said first and second tabs are hook fasteners, and said engagement of said first and second flaps with said first and second tabs permit said disposable undergarment to be worn by wearers having variations in weight of over 20 pounds.

14. The disposable undergarment of claim 10 wherein each of first and second flaps is capable of stretching in a first direction and in a second direction, where said second direction is perpendicularly aligned to said first direction, and said stretch in said second direction is no more than 90% stretch.

15. The disposable undergarment of claim 10 wherein each of said first and second flaps is a stretchable loop material having two types of threads including a first spandex thread covered with a texturized nylon and a second polyester thread and said stretchable loop material can stretch 220% in one direction.

16. The disposable undergarment of claim 15 wherein the said stretchable loop material is a knitted fabric including spandex elastic yarn, and said stretchable loop material is textured to increase its ability to engage with a hook fastener.

17. The disposable undergarment of claim 15 wherein said stretchable loop material is a stitch bonded fabric including at least one textured thread, and said stretchable loop material is textured to increase its ability to engage with a hook fastener.

18. The disposable undergarment of claim 15 wherein said stretchable loop material is subjected to a heat treatment prior to being incorporated into said disposable undergarment.

19. A method of affixing a disposable undergarment to a body of a wearer, said method comprising the steps of:
a) utilizing a disposable undergarment having a main body with an absorbent core, a first longitudinal side and a second longitudinal side, each of said first and second longitudinal sides being located laterally outboard of said absorbent core, said main body having a liquid permeable top sheet which contacts a wearer's body and a liquid impermeable backsheet spaced away from said wearer's body, a front waist portion and a rear waist portion, said front and rear waist portions configured to be positioned on a wearer's body under and away from a waist region of said wearer's clothing; first and second elastic extensions each having a proximal end and a distal end, said first elastic extension connected at said proximal end to said first longitudinal side of said main body and said second elastic extension connected at said proximal end to said second longitudinal side of said main body, each of said first and second elastic extensions positioned from a top edge of said rear waist portion to a center of said main body; a first flap and a second flap, said first flap connected to said distal end of said first elastic extension and said second flap connected to said distal end of said second elastic extension, said first and second flaps extending in a transverse direction away from said main body, each of said first and second flaps having a body facing surface which faces said wearer's body; and each of said first and second flaps are constructed of a stretchable loop material that can stretch in two directions and to 220% in a single direction, a first tab and a second tab, said first tab connected to said first longitudinal side of said main body and said second tab connected to said second longitudinal side of said main body, said first tab having a first attachment area with a plurality of engaging elements and said second tab having a second attachment area with a plurality of engaging elements, each of said first and second tabs longitudinally extending from an edge of said front waist portion toward said center of said main body, and said plurality of engaging elements on said first and second tabs are engagable with said first and second flaps to secure said disposable undergarment around a wearer's waist and under and away from a waist region of said wearer's clothing;
b) holding said liquid permeable top sheet of said main body of said disposable undergarment against the body of said wearer;
c) stretching said first elastic extensions around one of the hips of said wearer and pulling said first flap into an overlapping position with said first tab, and engaging said first flap with said first attachment area formed on said first tab; and
d) stretching said second elastic extensions around the other hip of said wearer and pulling said second flap into an overlapping position with said second tab, and engaging said second flap with said second attachment area formed on said second tab such that said disposable undergarment is secured around said wearer's waist and under and away from a waist region of said wearer's clothing.

20. The method of claim 19 wherein said first and second flaps are configured such that said body facing surface of each of said first and second flaps can be directly and releasably connected to said first and second attachment areas formed on said first and second tabs, at any location along said first and second flaps.

21. A method of disposing or transporting a soiled disposable undergarment, said method comprising the steps of:
a) utilizing a disposable undergarment having a main body with an absorbent core, a first longitudinal side and a second longitudinal side, each of said first and second longitudinal sides being located laterally outboard of said absorbent core, said main body having a liquid permeable top sheet which contacts a wearer's body and a liquid impermeable backsheet spaced away from said wearer's body, a front waist portion and a rear waist portion, said front and rear waist portions configured to be positioned on a wearer's body under and away from a waist region of said wearer's clothing; first and second elastic extensions each having a proximal end and a distal end, said first elastic extension connected at said proximal end to said first longitudinal side of said main body and said second elastic extension connected at said proximal end to said second longitudinal side of said main body, each of said first and second elastic extensions positioned from a top edge of said rear waist portion to a center of said main body; a first flap and a second flap, said first flap connected to said distal end of said first elastic extension and said second flap connected to said distal end of said second elastic extension, said first and second flaps extending in a transverse direction away from said main body, and each of said first and second flaps having a body facing surface which faces said wearer's body; and each of said first and second flaps are constructed of a stretchable loop material that can stretch in two directions and to 220% in a single direction, a first tab and a second tab, said first tab connected to said first longitudinal side of said main body and said second tab connected to said second longitudinal side of said main body, said first tab having a first attachment area with a plurality of engaging elements and said second tab having a second attachment area with a plurality of engaging elements, each of said first and second tabs longitudinally extending from an edge of said front waist portion toward said center of said main body, and said plurality of engaging elements on said first and second tabs are engagable with said first and second flaps to secure said disposable undergarment around a wearer's waist and under and away from a waist region of said wearer's clothing
b) releasing a first and second flaps from said first and second attachment areas formed on said first and second tabs at said front waist region of said disposable undergarment;
c) allowing said first and second elastic extensions to contract towards said main body;
d) rolling or folding said front waist portion inward and backward toward said rear waist portion of said disposable undergarment; and
e) tying said first and second flaps around said rolled or folded front waist portion to encase said rolled or folded front waist portion between said first and second flaps.

22. The disposable undergarment of claim 10 wherein said first and second flaps are constructed from a stretchable loop material which can stretch 190% in one row and 60% in a second row.

23. The disposable undergarment of claim 22 wherein said stretchable loop material is a knitted fabric.

24. The disposable undergarment of claim 22 wherein said stretchable loop material is a stitch bonded fabric.

25. The disposable undergarment of claim 10 wherein said undergarment is a low rise product.

26. The disposable undergarment of claim 10 wherein each of said first and second flaps has a height measured along a length of said main body and a width measured perpendicular to said height, and said length is at least twice said width.

27. The disposable undergarment of claim 10 wherein when said undergarment is worn about a waist of said wearer and said main body occupies about a third of said circumferential waist.

28. The disposable undergarment of claim 10 wherein said first flap can be secured to said plurality of engaging elements of said first tab at any location, and said second flap can be secured to said plurality of engaging elements of said second tab at any location.

29. The disposable undergarment of claim 10 wherein for a wearer having a small profile, said distal ends of said first and second flaps can extend past said first and second attachment areas of said first and second tabs to enable said undergarment to form a tight fit around said wearer's waist.
